(19) United States
(12) Patent Application Publication
(10) Pub. No.: US 2021/0386403 A1
(43) Pub. Date: Dec. 16, 2021

(54) ULTRASOUND OBSERVATION DEVICE, OPERATING METHOD FOR ULTRASOUND OBSERVATION DEVICE, AND COMPUTER READABLE RECORDING MEDIUM

(71) Applicant: OLYMPUS CORPORATION, Tokyo (JP)

(72) Inventor: Tatsuya Miyake, Tokyo (JP)

(73) Assignee: OLYMPUS CORPORATION, Tokyo (JP)

(21) Appl. No.: 17/462,542

(22) Filed: Aug. 31, 2021

Related U.S. Application Data
(63) Continuation of application No. PCT/JP2019/010396, filed on Mar. 13, 2019.

(51) Int. Cl. A61B 8/08; G06K 9/62; A61B 8/00

(52) U.S. Cl. CPC A61B 8/485; G06K 9/6202; A61B 8/08; A61B 8/5246; A61B 8/5223; A61B 8/54

(57) ABSTRACT

An ultrasound observation device is configured to acquire an echo signal which is obtained by converting, to an electrical signal, ultrasound received by an ultrasound probe by using a first measurement method that utilizes displacement of an observation target to measure elasticity information of the observation target, and a second measurement method that utilizes a shear wave generated by the observation target to measure the elasticity information of the observation target. The ultrasound observation device includes a processor configured to control the ultrasound observation device and the ultrasound probe, and the processor is configured to control the ultrasound probe to execute the second measurement method based on the echo signal obtained using the first measurement method.

This application is a continuation of International Application No. PCT/JP2019/010396, filed on Mar. 13, 2019, the entire contents of which are incorporated herein by reference.

### BACKGROUND

The present disclosure relates to an ultrasound observation device, an operating method for the ultrasound observation device, and a computer readable recording medium.

In the related art, ultrasound elastography has been known as technique for diagnosing observation targets using ultrasound. Ultrasound elastography is a technique that utilizes the fact that the stiffness of biological tissue varies according to disease progression and so forth. In this technique, an elasticity image is generated which is obtained by imaging elasticity information relating to the stiffness (elastic properties) of the biological tissue by coloring, as a reference value, the average value of the displacement amount of the biological tissue in a predetermined region of interest (ROI). In ultrasound elastography, a physician or other operator sets the region of interest according to what is being observed.

The strain method and the shear wave method are known as methods for measuring the elastic properties of tissue. The strain method measures elastic properties either by the operator applying pressure or by using displacement due to biological motion such as pulsation. The shear wave method uses a push pulse to generate a shear wave with respect to the biological tissue, and uses the velocity of the shear wave to measure elastic properties.

While the strain method exhibits high real-time performance, this method makes a quantitative evaluation difficult and affords low reproducibility. In contrast, the shear wave method is favorable for a quantitative evaluation and affords high reproducibility. Combining these techniques to measure elastic properties enables quantitative measurements in real time and with high reproducibility.

### SUMMARY

According to one aspect of the present disclosure, there is provided an ultrasound observation device configured to acquire an echo signal which is obtained by converting, to an electrical signal, ultrasound received by an ultrasound probe by using a first measurement method that utilizes displacement of an observation target to measure elasticity information of the observation target, and a second measurement method that utilizes a shear wave generated by the observation target to measure the elasticity information of the observation target, the ultrasound observation device including a processor configured to control the ultrasound observation device and the ultrasound probe, wherein the processor is configured to control the ultrasound probe to execute the second measurement method based on the echo signal obtained using the first measurement method.

According to another aspect of the present disclosure, there is provided an ultrasound observation device configured to acquire an echo signal which is obtained by converting, to an electrical signal, ultrasound received by an ultrasound probe by using a first measurement method that utilizes displacement of an observation target to measure elasticity information of the observation target, and a second measurement method that utilizes a shear wave generated by the observation target to measure the elasticity information of the observation target, the ultrasound observation device including a processor configured to control the ultrasound observation device and the ultrasound probe, wherein the processor is configured to: determine whether or not a reference condition of the first measurement method is fulfilled by using a plurality of temporally preceding or subsequent images based on the echo signal, which are two images based on the echo signal obtained using the first measurement method; determine, in a case where it is determined that the reference condition is fulfilled, whether or not an execution condition of the second measurement method is fulfilled by using a plurality of temporally preceding or subsequent images based on the echo signal, which are a plurality of images based on the echo signal obtained using the first measurement method; and control the ultrasound probe to execute the second measurement method in a case where it is determined that the execution condition is fulfilled.

The above and other features, advantages and technical and industrial significance of this disclosure will be better understood by reading the following detailed description of presently preferred embodiment of the disclosure, when considered in connection with the accompanying drawings.

### BRIEF DESCRIPTION OF THE DRAWINGS

FIG. 1 is a block diagram illustrating the configuration of an ultrasound observation system provided with an ultrasound observation device according to an embodiment;

FIG. 2 is a diagram illustrating processing performed by a time variation determination unit of the ultrasound observation device according to the embodiment;

FIG. 3 is a flowchart providing an overview of processing performed by the ultrasound observation device according to the embodiment;

FIG. 4 is a diagram illustrating an example of an elasticity image displayed by a display device of the ultrasound observation system according to the embodiment;

FIG. 5 is a diagram illustrating an example of an elasticity image displayed by the display device of the ultrasound observation system according to the embodiment;

FIG. 6 is a diagram illustrating an example of a displacement time variation based on measurement results obtained using the strain method;

FIG. 7 is a diagram illustrating an example of a displacement time variation based on measurement results obtained using the strain method;

FIG. 8 is a diagram illustrating processing performed by a matching degree determination unit of the ultrasound observation device according to the embodiment;

FIG. 9 is a diagram illustrating processing performed by a displacement amount determination unit and a movement amount determination unit of the ultrasound observation device according to the embodiment;

FIG. 10 is a flowchart providing an overview of processing performed by the ultrasound observation device according to the embodiment;

FIG. 11 is a diagram illustrating an example of a display image displayed by the display device of the ultrasound observation system according to the embodiment; and

FIG. 12 is a diagram illustrating an example of a display image displayed by the display device of the ultrasound observation system according to the embodiment.

### DETAILED DESCRIPTION

An embodiment of an ultrasound observation device (ultrasound imaging system), an operating method for the ultrasound observation device, and a computer readable recording medium storing an operating program for the ultrasound observation device will be described hereinbelow with reference to the drawings. Note that the present disclosure is not limited to or by such embodiment. The present disclosure may be generally applied to an ultrasound observation device that is capable of performing a diagnosis using ultrasound elastography.

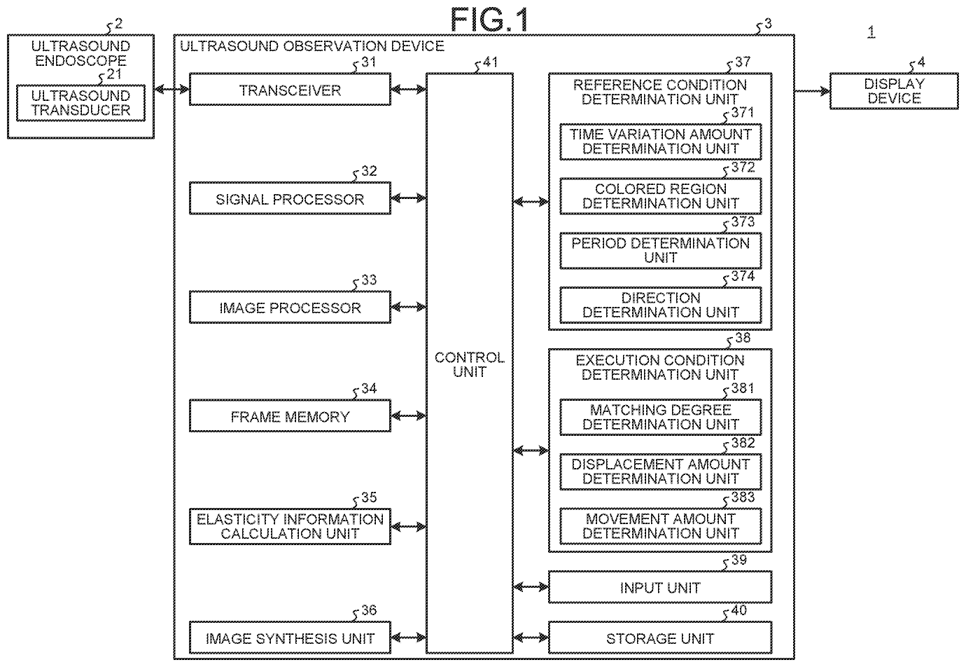

FIG. 1 is a diagram schematically illustrating the configuration of an ultrasound diagnosis system provided with an ultrasound observation device according to an embodiment. An ultrasound diagnosis system 1, which is illustrated in FIG. 1, includes: an ultrasound endoscope 2 that transmits ultrasound to a test subject constituting an observation target and receives ultrasound reflected by the test subject; an ultrasound observation device 3 that generates an ultrasound image based on an ultrasound signal acquired by the ultrasound endoscope 2; and a display device 4 that displays the ultrasound image generated by the ultrasound observation device 3.

The ultrasound endoscope 2 has, at the distal end thereof, an ultrasound transducer 21 that converts an electrical pulse signal received from the ultrasound observation device 3 to ultrasound pulses (acoustic pulses) and projects said pulses onto a test subject, converts an ultrasound echo reflected by the test subject into an electrical echo signal (ultrasound signal) represented by a voltage variation, and outputs said echo signal. The ultrasound transducer 21 is realized by a convex-type transducer. However, the ultrasound transducer 21 may also be configured so as to be realized by a radial-type or a linear-type transducer or the like. The ultrasound endoscope 2 may be such that the ultrasound transducer 21 is made to perform scanning mechanically, or may be such that same is made to perform scanning electronically by providing a plurality of elements in an array as the ultrasound transducer 21, electronically switching the elements involved in transmission and reception, and applying a delay to the transmission and reception of each element.

The ultrasound endoscope 2 normally has an imaging optical system and an image sensor and is capable of being inserted into the digestive tract (esophagus, stomach, duodenum, large intestine) or respiratory organs (trachea, bronchus) of the test subject to image the digestive tract, respiratory organs, and surrounding organs (pancreas, liver, gall bladder, bile duct, biliary tract, lymph nodes, mediastinal organs, blood vessels, and so forth). The ultrasound endoscope 2 also has a light guide for guiding illumination light that is projected onto the test subject during imaging. The distal end of the light guide reaches as far as the distal end of the part of the ultrasound endoscope 2 inserted into the test subject, while the proximal end of the light guide is connected to a light source device for generating the illumination light.

The ultrasound observation device 3 is provided with a transceiver 31, a signal processor 32, an image processor 33, a frame memory 34, an elasticity information calculation unit 35, an image synthesis unit 36, a reference condition determination unit 37, an execution condition determination unit 38, an input unit 39, a storage unit 40, and a control unit 41.

The transceiver 31 is electrically connected to the ultrasound endoscope 2, transmits a transmission signal (pulse signal) formed of high-voltage pulses based on a predetermined waveform and transmission timing to the ultrasound transducer 21, receives an echo signal, which is an electrical reception signal from the ultrasound transducer 21, generates digital high-frequency (radio frequency (RF)) signal data (hereinafter referred to as RF data), and outputs this signal data to the signal processor 32.

The frequency band of the pulse signal transmitted by the transceiver 31 may be in a wide band that substantially covers the linear response frequency band of the electroacoustic conversion of the pulse signal of the ultrasound transducer 21 to ultrasound pulses.

The transceiver 31 also has functions for transmitting various control signals outputted by the control unit 41 to the ultrasound endoscope 2 and for receiving various information including an identification ID from the ultrasound endoscope 2 and transmitting this information to the control unit 41.

Furthermore, upon acquiring control information from the control unit 41 to the effect that elastography is to be performed, the transceiver 31 transmits, to the ultrasound transducer 21, a transmission signal (pulse signal) formed of high-voltage pulses based on waveforms and transmission timing that serve to obtain a B-mode image and an elastography-related image (elasticity image). More specifically, the transceiver 31 superimposes an elastography pulse on a pulse for B-mode image acquisition, for example. The transceiver 31 transmits ultrasound a plurality of times in the same direction and receives a plurality of reflected echo signals, thereby acquiring elastography echo signals. Upon receiving the elastography echo signals, the transceiver 31 generates elastography RF data and outputs same to the signal processor 32. In this embodiment, the transceiver 31 causes the ultrasound transducer 21 to execute the transmission and reception of ultrasound according to either the strain method or the shear wave method, under the control of the control unit 41.

The signal processor 32 generates digital B-mode reception data based on the RF data received from the transceiver 31. More specifically, the signal processor 32 subjects the RF data to well-known processing such as a bandpass filter, envelope detection, and logarithmic transformation, and generates digital B-mode reception data. In logarithmic conversion, the common logarithm for the amount of RF data divided by the reference voltage is taken and expressed using decibel values. The B-mode reception data is formed from a plurality of line data obtained by aligning the amplitudes or the intensities of the reception signals representing the intensities of reflection of the ultrasound pulses, along the direction in which the ultrasound pulses are transmitted/received (the depth direction). The signal processor 32 outputs one frame's worth of the generated B-mode reception data to the image processor 33.

Furthermore, the signal processor 32 generates elastography reception data based on the elastography RF data received from the transceiver 31. More specifically, the signal processor 32 uses RF data in the same direction to calculate, for each predetermined depth, the variation in the amplitude or intensity of the reception signals representing the intensities of reflection of the ultrasound pulses, and generates a sound line (line data) which has the calculated variation amount. The elastography reception data is formed from a plurality of line data obtained by aligning the amounts of variation in the amplitudes or the intensities of the reception signals representing the intensities of reflection of the ultrasound pulses, along the direction in which the ultrasound pulses are transmitted/received (the depth direction). The signal processor 32 is realized using a central processing unit (CPU) and various computation circuits, or the like.

The image processor 33 generates B-mode image data based on the B-mode reception data received from the signal processor 32. The image processor 33 generates B-mode image data by performing signal processing using known techniques such as scan converter processing, gain processing, and contrast processing on the B-mode reception data outputted from the signal processor 32, as well as data thinning, or the like, which corresponds to the data step width defined according to the display range of the image on the display device 4. In scan converter processing, the scanning direction of the B-mode reception data is converted from the ultrasound scanning direction to the display direction of the display device 4. An ultrasound image constituting a B-mode image is a grayscale image in which the values of R (red), G (green), and B (blue), which are variables when the RGB color system is adopted as the color space, are matched. Note that the image generated by the image processor 33 is larger than the display region which the display device 4 is capable of displaying. In other words, the B-mode image displayed by the display device 4 is a portion of the B-mode image generated by the image processor 33.

In addition, the image processor 33 generates elastography image data in the region of interest (ROI) set by the control unit 41 (described subsequently) based on the elasticity information calculated by the elasticity information calculation unit 35 (described below). Specifically, the image processor 33 generates elastography image data by applying pseudo color information to each depth position according to the relative variation amount in the set region of interest. The color information is elasticity information that indicates the stiffness of the observation target in each position, and is represented by a color that is determined relative to the percentage of the variation amount in the region of interest. Thus, in this embodiment, a region where color information is added to the image data is referred to as a colored region.

The image processor 33 performs a coordinate transformation that rearranges the B-mode reception data from the signal processor 32 and the elasticity information from the elasticity information calculation unit 35 so that the scanning range may be represented spatially correctly, and then performs interpolation processing between the B-mode reception data and the elastography reception data to fill in the gaps between the B-mode reception data to generate B-mode image data and elastography image data. The image processor 33 is realized using a CPU and various computation circuits, or the like.

The frame memory 34 is realized using a ring buffer, for example, and stores one frame of B-mode image data generated by the image processor 33 in order of acquisition time. The frame memory 34 may also store a plurality of frames of B-mode image data in chronological order. In this case, when the frame memory 34 runs out of capacity (when a predetermined number of frames of B-mode image data are stored), a predetermined number of frames of the latest B-mode image data are stored in chronological order by overwriting the oldest B-mode image data with the latest B-mode image data.

Furthermore, the elasticity information calculation unit 35 calculates elasticity information of the observation target in a preset region in the ultrasound image based on the elastography reception data received from the signal processor 32. The preset region is a region (region of interest) designated in the ultrasound image by the operator, and the elasticity information calculation unit 35 calculates the elasticity information in each position in the region of interest. However, the preset region may also be the entirety of the ultrasound image. Elasticity information here refers to, for example, the displacement amount and the modulus of elasticity. The elasticity information calculation unit 35 according to this first embodiment is described hereinbelow as calculating the displacement amount. The elasticity information calculation unit 35 is realized using a CPU and various computation circuits, or the like.

The image synthesis unit 36 generates an image by combining the elastography image data of the region of interest with the B-mode image data generated by the image processor 33. Specifically, the image synthesis unit 36 generates an image (elasticity image) in which color information corresponding to the elasticity information calculated by the elasticity information calculation unit 35 for the ultrasound image is combined with a B-mode image together with a frame of the region of interest which may be identified by a dashed, dotted, or solid line, or the like. The image synthesis unit 36 is realized using a CPU and various computation circuits, or the like.

The reference condition determination unit 37 determines whether or not the conditions for transitioning to the shear wave method are fulfilled from the elasticity information obtained using the strain method. The reference condition determination unit 37 has a time variation amount determination unit 371, a colored region determination unit 372, a period determination unit 373, and a direction determination unit 374. The reference condition determination unit 37 determines whether or not the conditions for transitioning to the shear wave method are fulfilled based on each of the determination results of the time variation amount determination unit 371, the colored region determination unit 372, the period determination unit 373, and the direction determination unit 374. When it is determined from the determination result of the time variation amount determination unit 371 and from the determination results of any of the colored region determination unit 372, the period determination unit 373, and the direction determination unit 374, that the reference condition for transitioning to the shear wave method is fulfilled, the reference condition determination unit 37 sets the image determined to fulfill the reference condition as the reference image. The reference condition determination unit 37 is realized using a CPU and various computation circuits, or the like.

The time variation amount determination unit 371 determines whether or not an image fulfilling the conditions for implementing the determination of the reference conditions has been obtained based on the time variation amount. When there is an input for implementing the strain method, the time variation amount determination unit 371 determines whether the operator is searching for an observation target or has found an observation target and is performing detailed observation, by using temporally preceding or subsequent elasticity images (having different acquisition times) to calculate the time variation amount. The time variation amount determination unit 371 calculates the time variation amount by using known methods such as pattern matching, statistical values calculated from histograms of luminance values, and the differential from the luminance values to determine the matching degree between the elasticity images. A reference condition determination unit 37A moves to processing to determine the aforementioned reference condition if the time variation amount determination unit 371 determines that this time variation amount is less than the reference time variation amount.

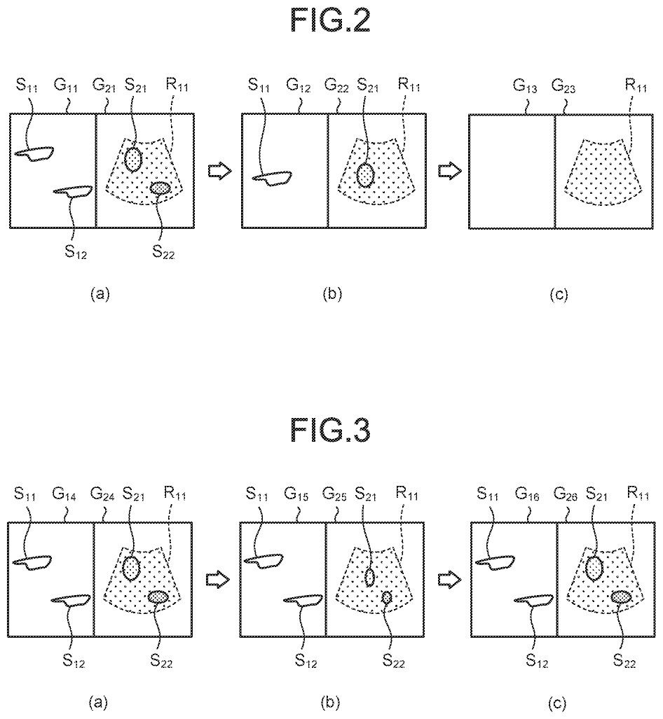

FIGS. 2 and 3 are diagrams illustrating processing performed by a time variation determination unit of the ultrasound observation device according to the embodiment. The images illustrated in (a), (b), and (c) of FIG. 2 are examples of images which are each acquired at different times while the operator is searching for an observation target. The images illustrated in (a), (b), and (c) of FIG. 3 are examples of images which are each acquired at different times in the state of finding the observation target and performing detailed observation. FIGS. 2 and 3 schematically illustrate images with B-mode images G₁₁ to G₁₆ (grayscale images) displayed on the left side and elasticity images G₂₁ to G₂₆ (images with color information) displayed on the right side. In FIG. 2, the tissue S₁₂ in the B-mode image G₁₁ in FIG. 2(a) has disappeared in the B-mode image G₁₂ in FIG. 2(b). Furthermore, the tissues S₁₁ and S₁₂ in the B-mode image G₁₁ in FIG. 2 (a) have disappeared in the B-mode image G₁₃ in FIG. 2 (c). If the tissue in the image varies or disappears over time, the time variation amount in the image position to be acquired is large, and in light of this condition, it is determined that the operator is searching for an observation target. In this case, the elasticity images G₂₁ to G₂₃ display the elasticity information S₂₁ and S₂₂ corresponding to the tissues S₁₁ and S₁₂.

In contrast, in FIG. 3, the tissues S₂₁ and S₂₂ that were captured in the B-mode image G₁₄ in FIG. 3(a) are also present in the B-mode image G₁₅ in FIG. 3(b) and in the B-mode image G₁₆ in FIG. 3(c). Thus, if the same tissue is captured irrespective of time variation, the time variation amount is small, and the time variation amount determination unit 371 determines that the operator has found the observation target and is making a detailed observation. In this case, the elasticity images G₂₄ to G₂₆ display the elasticity information S₂₁ and S₂₂ corresponding to the tissues S₁₁ and S₁₂.

The colored region determination unit 372 calculates the surface area of the elasticity information calculation region in the region of interest by using the elasticity information calculated by the elasticity information calculation unit 35, and determines whether or not the surface area is greater than or equal to the reference surface area. The reference surface area is a preset surface area, and is set as a percentage of the surface area of the region of interest, for example.

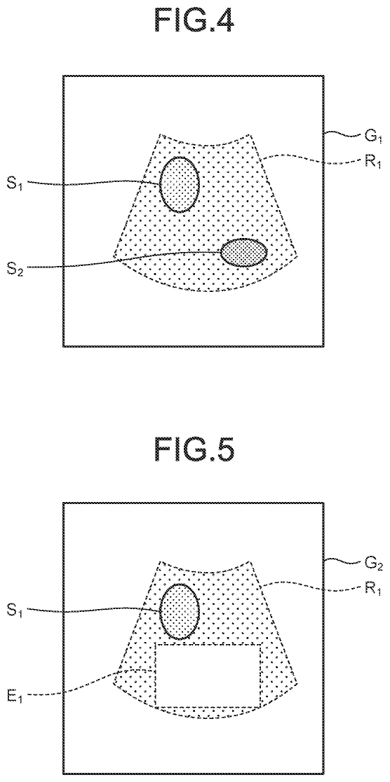

FIGS. 4 and 5 are diagrams illustrating an example of an elasticity image displayed by the display device of the ultrasound observation system according to the first embodiment. The elasticity images G₁ and G₂ illustrated in FIGS. 4 and 5 are images obtained using the strain method and are images obtained by superimposing colors corresponding to the elasticity information within the region of interest R₁ set in the aforementioned B-mode image. For example, color is applied specifically to the tissues S₁ and S₂, which have relatively different stiffness from other body parts. In FIGS. 4 and 5, the regions where color is applied are illustrated with hatching. The elasticity information is assigned a preset color according to the variation amount or the modulus of elasticity of each pixel, for example. Further, the error region E₁ illustrated in FIG. 5 is a region in which elasticity information (the variation amount) could not be calculated due to noise or the like.

The elasticity image G₁ illustrated in FIG. 4 has color superimposed in all of the regions of interest R₁. In contrast, the elasticity image G₂ illustrated in FIG. 5 has color superimposed in the region of interest R₁, except the error region E₁. For example, when the elasticity image G₂ is obtained, the colored region determination unit 372 determines whether the surface area of the region other than the error region E₁ (the colored region) is greater than or equal to the reference surface area set for the region of interest R₁, for example.

The period determination unit 373 determines whether or not the displacement amount in the region of interest R₁ is periodic. Using the elasticity information calculated by the elasticity information calculation unit 35, the period determination unit 373 calculates the displacement amount, for each frame, in the region of interest, and calculates the time variation of the variation amount. The period determination unit 373 determines whether or not the volatility of the time period in which the displacement amount becomes zero is less than or equal to the reference volatility which is preset. The reference volatility is preset and is set at 30%, for example. Note that the period determination unit 373 may also detect the time period in which the displacement amount peaks to determine volatility.

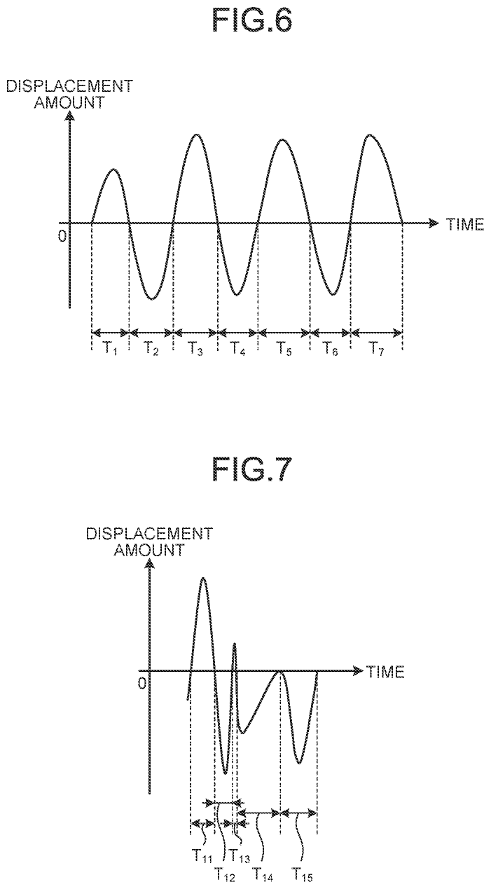

FIGS. 6 and 7 are diagrams illustrating an example of a displacement time variation based on measurement results obtained using the strain method. The period determination unit 373 respectively calculates the time period from the time when the displacement amount becomes zero to the next time same reaches zero (in FIG. 6, the time periods T₁, T₂, . . . , T₇, and in FIG. 7, the time periods T₁₁, T₁₂, . . . , T₁₅), and then calculates the volatility ((T_{L}−T_{S})/T_{S}) between the shortest time period (which is T_{S}) and the longest time period (which is T_{L}). The period determination unit 373 determines whether or not the volatility determined is less than or equal to the reference volatility. When there is variation in the volatility calculated from the displacement amount, the period determination unit 373 determines that the variation is not periodic in the case of the time variation illustrated in FIG. 7 by way of an example.

The direction determination unit 374 determines whether or not the displacement direction in the region of interest R₁ is perpendicular to the scanning direction of the ultrasound. The direction determination unit 374 determines the displacement direction from the elasticity information in each scanning position, and detects the orientation of the determined displacement direction relative to the scanning direction. The direction determination unit 374 determines whether or not the detected displacement direction is perpendicular to the scanning direction of the ultrasound.

Based on the determination results of the colored region determination unit 372, the period determination unit 373, and the direction determination unit 374, the reference condition determination unit 37 determines whether or not the conditions for transitioning to the shear wave method are fulfilled. More specifically, the reference condition determination unit 37 determines that the conditions for transitioning to the shear wave method are fulfilled if at least one of the following is fulfilled: when the surface area of the elasticity information calculation region is determined by the colored region determination unit 372 to be greater than or equal to the reference surface area, when the displacement amount in the region of interest R₁ is determined by the period determination unit 373 to be periodic, and when the displacement direction in the region of interest R₁ is determined by the direction determination unit 374 to be perpendicular to the scanning direction.

When the reference conditions are fulfilled in the strain method, the execution condition determination unit 38 determines, based on the reference image, whether or not the conditions (execution conditions) for executing the shear wave method are fulfilled. The execution condition determination unit 38 has a matching degree determination unit 381, a displacement amount determination unit 382, and a movement amount determination unit 383. The execution condition determination unit 38 determines whether or not the conditions for executing the shear wave method are fulfilled based on the determination results of the matching degree determination unit 381 and the determination results of the displacement amount determination unit 382 or the movement amount determination unit 383. The execution condition determination unit 38 is realized using a CPU and various computation circuits, or the like.

The matching degree determination unit 381 calculates the matching degree of the image to be determined with respect to a reference image, and compares the calculated matching degree with the reference matching degree. The reference image and the image to be determined are not identical, rather, same are temporally preceding or subsequent images, with the image to be determined being temporally subsequent to the reference image. The matching degree is calculated in the same way as the foregoing time variation amount determination unit 371, using known methods such as pattern matching, statistical values calculated from histograms of luminance values, and the amount of difference from the luminance values. In the case where the higher the match, the larger the value of the matching degree, the matching degree determination unit 381 determines whether the matching degree with respect to the reference image is greater than or equal to the reference matching degree. The reference image in this first embodiment is the image (B-mode image or elasticity image) that is determined by the reference condition determination unit 37 to fulfill the conditions for transitioning to the shear wave method. In this first embodiment, the target region for the matching degree determination is within the region of interest set for each image. The entire image may also be used as the determination target.

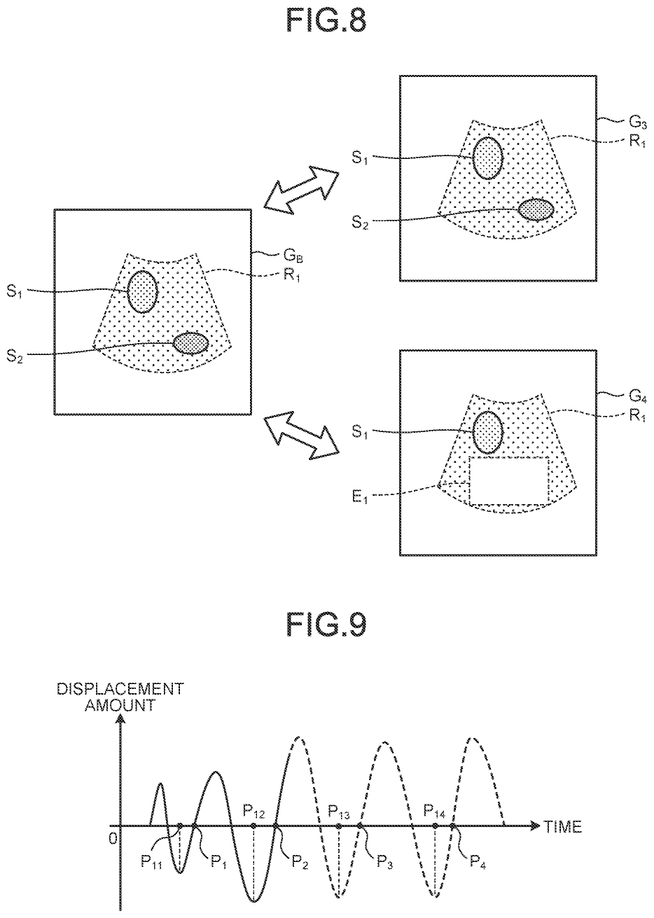

FIG. 8 is a diagram illustrating processing performed by the matching degree determination unit of the ultrasound observation device according to the first embodiment. In FIG. 8, an elasticity image is illustrated as an example. The matching degree determination unit 381 calculates the degree of matching with the reference image G_{B} for the elasticity images G₃ and G₄. For example, for the elasticity image G₃, which contains the same tissues S₁ and S₂ as tissues S₁ and S₂ in the reference image G₈, the matching degree determination unit 381 determines that the matching degree with the reference image G_{B} is greater than or equal to the reference matching degree, and determines that, for the elasticity image G₄, which does not show the tissue S₂ in the reference image G₈, the matching degree with the reference image G₈ is less than the reference matching degree.

FIG. 9 is a diagram illustrating processing performed by a displacement amount determination unit and a movement amount determination unit of the ultrasound observation device according to the first embodiment.

The displacement amount determination unit 382 detects a peak in the displacement amount from the time variation of the displacement amount, and uses this peak as the tissue displacement amount to determine whether or not the tissue displacement amount is less than the reference displacement amount. When the amount of tissue displacement is less than the reference displacement amount, the displacement amount determination unit 382 predicts the timing at which the displacement amount becomes zero. A “displacement amount peak” here refers to the timing at which the displacement amount varies the most in the positive direction, and, in this case, is the timing at which the displacement amount transitions from the negative direction to the positive direction. In other words, positions for which the displacement amount is at zero and at which the displacement amount transitions from negative to positive (the positions P₁, P₂, P₃, and P₄ in FIG. 9). The displacement amount determination unit 382, for example, predicts the timing of positions P₃ and P₄ by detecting positions P₁ and P₂ when a displacement amount is not obtained at and beyond positions P₁ and P₂ in FIG. 9. By acquiring images at the predicted positions P₃ and P₄, an image that is minimally affected by displacement may be obtained. The reference displacement amount is set to the displacement amount that is permissible in performing the shear wave method. Note that the displacement amount determination unit 382 may also detect the timing when the displacement amount transitions from the positive direction to the negative direction as the displacement peak.

The movement amount determination unit 383 calculates the maximum value of the difference of a plurality of periods from the time variation (period) of the displacement, uses this maximum value as the tissue movement amount, and determines whether or not the tissue movement amount is less than the reference movement amount. When the tissue movement amount is less than the reference movement amount, the movement amount determination unit 383 predicts the timing of a displacement peak. The “displacement peak” here refers to the timing when the displacement amount is the largest, and in this first embodiment, is the position where the displacement turns from the negative direction to the positive direction (positions P₁₁, P₁₂, P₁₃, and P₁₄ in FIG. 9). For example, when the displacement amount at and beyond positions P₁₁ and P₁₂ is not obtained, the movement amount determination unit 383 detects positions P₁₁ and P₁₂ and predicts the timing of positions P₁₃ and P₁₄. The reference movement amount is set to the movement amount that is permissible in executing the shear wave method. Note that the movement amount determination unit 383 may also detect, as the displacement peak, the timing at which the displacement amount transitions from the positive direction to the negative direction.

Here, the foregoing amount of tissue displacement and the amount of tissue movement indicate the amount of variation in mutually different directions. For example, the amount of tissue displacement is the amount of variation in the direction in which the tissue moves due to pulsation, and the amount of tissue movement is the amount of variation in the position of the tissue relative to the ultrasound endoscope 2 (ultrasound transducer 21).

The execution condition determination unit 38 determines whether or not the conditions for executing the shear wave method are fulfilled in light of the determination results of the matching degree determination unit 381 and the displacement amount determination unit 382 or the movement amount determination unit 383. Specifically, the execution condition determination unit 38 determines that the conditions for executing the shear wave method are fulfilled when the matching degree is determined by the matching degree determination unit 381 to be greater than or equal to the reference matching degree, and either when the timing at which the displacement amount becomes zero is predicted by the displacement amount determination unit 382, or when the timing at which the displacement peaks is predicted by the movement amount determination unit 383. When the conditions for executing the shear wave method are determined to be fulfilled by the execution condition determination unit 38, the control unit 41 transmits a push pulse to the ultrasound transducer 21 to execute the shear wave method.

The input unit 39 is realized using a user interface such as a keyboard, mouse, track ball, touch panel, and the like, and receives inputs of various information. The input unit 39 outputs the received information to the control unit 41. The input unit 39 receives inputs from the operator to set the region of interest to the desired region.

The storage unit 40 stores various programs for operating the ultrasound diagnosis system 1, and data including various parameters and so forth necessary for the operation of the ultrasound diagnosis system 1.

Furthermore, the storage unit 40 stores various programs, including an operating program for executing the operating method for the ultrasound diagnosis system 1. The operating program may also be recorded on a computer-readable recording medium such as a hard disk, flash memory, CD-ROM, DVD-ROM, flexible disk, or the like, to enable wide distribution. Note that the foregoing various programs may also be acquired by being downloaded via a communication network. The communication network here is realized, for example, by an existing public line network, a LAN (Local Area Network), a WAN (Wide Area Network), or the like, and may be wired or wireless.

The storage unit 40 with the foregoing configuration is realized using a ROM (Read Only Memory) in which various programs, or the like, are pre-installed, and a RAM (Random Access Memory), or the like, which stores computation parameters, data, and the like, for each processing.

The control unit 41 controls the entire ultrasound diagnosis system 1. The control unit 41 is realized using a CPU and various computation circuits, or the like, that have computation and control functions. The control unit 41 reads the information stored by the storage unit 40 from the storage unit 40 and performs centralized control of the ultrasound observation device 3 by executing various computation processing related to the operating method of the ultrasound observation device 3. The control unit 41 sets the region of interest for the ultrasound image based on the information inputted via the input unit 39. This region of interest corresponds to the region for calculating the foregoing elasticity information. Note that it is also possible to configure the control unit 41 using a CPU or the like common to the signal processor 32, image processor 33, elasticity information calculation unit 35, image synthesis unit 36, reference condition determination unit 37, and execution condition determination unit 38.

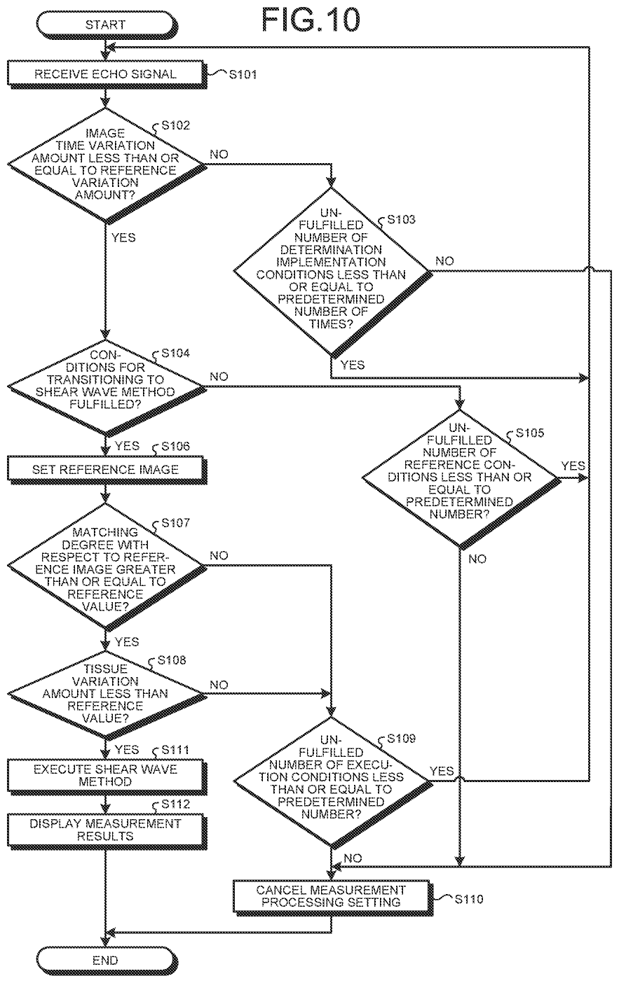

FIG. 10 is a flowchart illustrating an overview of processing executed by the ultrasound observation device 3 with the foregoing configuration. In FIG. 10, the mode for calculating the elasticity information is set, and the ultrasound transducer 21 receives ultrasound echoes using the strain method. First, the ultrasound observation device 3 receives, from the ultrasound endoscope 2, an echo signal as a result of measurement of the observation target by the ultrasound transducer 21 (step S101). The elasticity information calculation unit 35 calculates the elasticity information in the region of interest based on the received echo signal. At such time, each time the elasticity information is calculated, an elasticity image reflecting the latest elasticity information may also be displayed on the display device 4.

Upon receiving the echo signal from the ultrasound transducer 21, the time variation amount determination unit 371 calculates the time variation amount from the elasticity information (elasticity image) obtained using the strain method and with a different acquisition time and compares the time variation amount with the reference time variation amount (step S102). Here, if the time variation amount is less than the reference time variation amount (step S102: Yes), the time variation amount determination unit 371 moves to step 3104. In contrast, if the time variation amount is greater than the reference time variation amount (step S102: No), the time variation amount determination unit 371 moves to step S103.

In step S103, the reference condition determination unit 37 increases the number of unfulfilled determination implementation conditions by one and determines whether or not the number of unfulfilled determination implementation conditions is less than or equal to a predetermined number of times which is preset. If the number of unfulfilled times is determined to be less than or equal to the predetermined number of times (step S103: Yes), the reference condition determination unit 37 returns to step S101 and repeats the foregoing processing. On the other hand, upon determining that the number of unfulfilled times is greater than a predetermined number (step S103: No), the reference condition determination unit 37 moves to step S110. The number of unfulfilled times is preset according to the frame rate and processing speed. There are steps to determine the number of unfulfilled items in the following processing (steps S107 and S112), and the predetermined number of times set in these steps may be the same or different.

Upon receiving the echo signal from the ultrasound transducer 21, the reference condition determination unit 37 determines whether or not the conditions for transitioning to the shear wave method are fulfilled from the elasticity information obtained using the strain method (step S104).

In step S104, in the reference condition determination processing, the colored region determination unit 372, the period determination unit 373, and the direction determination unit 374 calculate the respective parameters and determine whether or not the predetermined conditions are fulfilled.

The colored region determination unit 372 calculates the surface area of the elasticity information calculation region in the region of interest by using the elasticity information calculated by the elasticity information calculation unit 35, and determines whether or not the surface area is greater than or equal to the reference surface area.

The period determination unit 373 determines whether or not the displacement amount in the region of interest (for example, the foregoing region of interest R₁) is periodic.

The direction determination unit 374 determines whether or not the displacement direction in the region of interest (for example, the foregoing region of interest R₁) is perpendicular to the scanning direction of the ultrasound.

More specifically, the reference condition determination unit 37 moves to step S106 when at least one of the following determination results is met: a determination result in which the surface area of the elasticity information calculation region is determined by the colored region determination unit 372 to be greater than or equal to the reference surface area, a determination result in which the displacement amount in the region of interest is determined by the period determination unit 373 to be periodic, and a determination result in which the displacement direction is determined by the direction determination unit 374 to be perpendicular to the scanning direction of the ultrasound, and it is determined the conditions for transitioning to the shear wave method are fulfilled (step S104: Yes).

In contrast, the reference condition determination unit 37 moves to step S105 when a determination result that the surface area of the elasticity information calculation region is determined by the colored region determination unit 372 to be less than the reference surface area, a determination result in which the displacement amount in the region of interest is determined by the period determination unit 373 to not be periodic, and a determination result in which the displacement direction is determined by the direction determination unit 374 to not be perpendicular to the scanning direction of the ultrasound are obtained, and determined that the conditions for transitioning to the shear wave method are not fulfilled (step S104: No).

In step S105, the reference condition determination unit 37 increases the number of unfulfilled reference conditions by one and determines whether the number of unfulfilled reference conditions is less than or equal to a predetermined number of times which is preset. Upon determining the number of unfulfilled times to be less than or equal to the predetermined number of times (step S105: Yes), the reference condition determination unit 37 returns to step S101 and repeats the foregoing processing. On the other hand, upon determining that the number of unfulfilled times is greater than the predetermined number of times (step S105: No), the reference condition determination unit 37 moves to step S110.

Furthermore, in step S106, the reference condition determination unit 37 determines that the reference conditions for transitioning to the shear wave method are fulfilled according to the determination results of any of the colored region determination unit 372, the period determination unit 373, and the direction determination unit 374, and sets an image which is determined as fulfilling the reference conditions as the reference image.

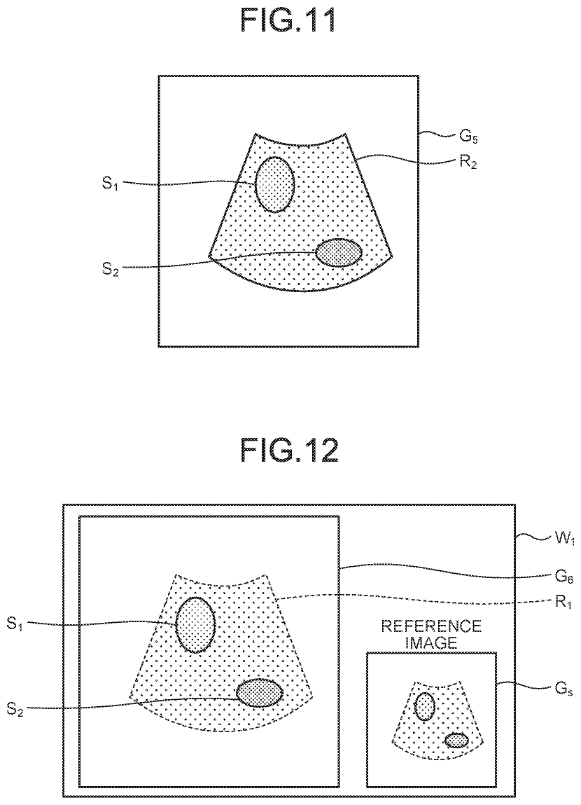

By modifying the display mode at such time, the operator may be notified that the reference conditions have been fulfilled. FIGS. 11 and 12 illustrate an example of a display image illustrated by the display device of the ultrasound observation system according to the first embodiment. For example, when the frame of the region of interest is displayed on the elasticity image, the displaying of the frame of the region of interest R₂ is modified in the elasticity image G₅ illustrated in FIG. 11. In the elasticity image G₅, the color of the frame is changed and the line type is changed to inform the operator that the reference conditions have been fulfilled. Further, the display screen W₁ illustrated in FIG. 12 displays the elasticity image G₆ as well as the elasticity image G₅ selected as the reference image. In the display screen W₁, the reference image is displayed to inform the operator that the reference conditions have been fulfilled. Note that these display modes may be modified through an operator input to the input unit 39.

In steps S107 and S108, which follow step S106, the execution condition determination unit 38 determines whether or not the conditions for executing the shear wave method are fulfilled. Note that, although this embodiment describes an example in which the processing of the displacement amount determination unit 382 is given priority, a setting may be made so that the movement amount determination unit 383 is given priority in processing, or a setting may be made so that only the determination unit set as the processing execution target performs processing.

In the execution condition determination processing, first, the matching degree determination unit 381 determines whether or not the matching degree with respect to the reference image is greater than or equal to the reference matching degree (step S107). The execution condition determination unit 38 transitions to step S108 when the matching degree determination unit 381 determines that the matching degree with respect to the reference image is greater than or equal to the reference matching degree (step S107: Yes). In contrast, the execution condition determination unit 38 transitions to step S109 when the matching degree determination unit 381 determines that the matching degree with respect to the reference image is less than the reference matching degree (step S107: No).

In step S108, the execution condition determination unit 38 determines whether or not the tissue variation amount is less than the reference value in the light of the determination results of the displacement amount determination unit 382 and the movement amount determination unit 383.

In step S108, the displacement amount determination unit 382 determines whether or not the foregoing tissue displacement amount is less than the reference displacement amount.

Furthermore, in step S108, the movement amount determination unit 383 determines whether or not the foregoing tissue movement amount is less than the reference movement amount.

When the execution condition determination unit 38 obtains the determination result determined by the displacement amount determination unit 382 that the tissue displacement amount is less than the reference displacement amount, or the determination result determined by the movement amount determination unit 383 that the tissue movement amount is less than the reference movement amount, and determines that the tissue movement amount is less than the reference value (step S108: Yes), the execution condition determination unit 38 detects the position where the displacement amount becomes zero and predicts the timing when the displacement amount becomes zero, or detects the peak of the displacement amount and predicts the timing at which the displacement peaks, and then moves to step S111. In contrast, when the execution condition determination unit 38 obtains the determination result determined by the displacement amount determination unit 382 that the tissue displacement amount is greater than or equal to the reference displacement amount from the time variation of the displacement amount, or the determination result determined by the movement amount determination unit 383 that the tissue movement amount is greater than or equal to the reference movement amount, and determines that the tissue movement amount is greater than or equal to the reference value (step S108: No), the execution condition determination unit 38 moves to step S109.

In step S109, the execution condition determination unit 38 increases the number of unfulfilled execution conditions by one, and determines whether or not the number of unfulfilled execution conditions is less than or equal to a predetermined number of times which is preset. Upon determining that the number of unfulfilled times is less than or equal to the predetermined number (step S109: Yes), the execution condition determination unit 38 returns to step S101 and repeats the foregoing process. On the other hand, upon determining that the number of unfulfilled times is greater than the predetermined number (step S109: No), the execution condition determination unit 38 moves to step S110.

In step S110, the control unit 41 cancels the measurement processing setting. When the measurement processing setting is released, the elasticity information calculation unit 35 ends the elasticity information calculation processing. After canceling the measurement processing setting, the control unit 41 ends the processing for the elasticity information calculation. At such time, the control unit 41 displays the B-mode image live on the display device 4, for example, according to the setting.

Furthermore, in step S111, upon determining that the conditions for executing the shear wave method are fulfilled by the processing of steps S107 and S108, the control unit 41 causes the shear wave method to be executed. The control unit 41 transmits push pulses to the ultrasound transducer 21 based on the timing at which the displacement amount predicted by the displacement amount determination unit 382 becomes zero, or the timing at which the displacement predicted by the movement amount determination unit 383 peaks.

In step S112 following step S111, the control unit 41 displays, on the display device 4, the measurement results relating to the elasticity information calculated by the elasticity information calculation unit 35 based on the echo signal obtained by the shear wave method in step S111, and displays an elasticity image or B-mode image.

According to one embodiment described hereinabove, when it is determined whether or not a condition serving as a reference is fulfilled in the strain method and the reference condition is fulfilled, the elasticity image fulfilling the reference condition is used to determine whether or not a condition for executing the shear wave method is fulfilled, and when the execution condition is fulfilled, the shear wave method is executed. According to this embodiment, when the necessary conditions for implementing each method are fulfilled, because elasticity information is acquired using the shear wave method, elastic properties may be measured properly by combining the strain method and the shear wave method.

Furthermore, according to this embodiment, before implementing the determination processing, it is determined whether the operator is in the process of finding the observation target or has specified the observation target in the light of the time variation amount of the elasticity image, and after such determination, the reference and execution conditions for executing the foregoing shear wave method are determined. Hence, such determination makes it possible to prevent, while the operator is searching for the observation target, unnecessary information such as a calculation processing of the elasticity information and the like is produced.

Furthermore, in this embodiment, the reference image for determining the execution condition is set as the elasticity image used for the reference condition, and hence the reference image may be easily set without relying on operator experience or the like.

Note that the determination processing for executing the foregoing shear wave method may also be designated as valid/invalid by the operator using the input unit 39. Also, a setting may also be made so that the foregoing determination processing is not performed when the region of interest is being set or re-set, that is, while the region of interest is moving.

Although modes for carrying out the present disclosure have been described thus far, the disclosure should not be limited only by the foregoing embodiment. The present disclosure may include various embodiments and so forth which have not been disclosed here. In the foregoing embodiment, an extracorporeal ultrasound probe, which irradiates ultrasound from the body surface of the test subject, may also be applied as an ultrasound probe. Extracorporeal ultrasound probes are usually used when observing abdominal organs (liver, gallbladder, bladder), breasts (especially mammary glands), and the thyroid gland.

Note that, in this embodiment, the measurement results obtained using the shear wave method are described as displayed. However, a single parameter may also be calculated from the measurement results of the strain method and the shear wave method and displayed as the measurement results.

As described hereinabove, the ultrasound observation device, the operating method for the ultrasound observation device, and the operating program for the ultrasound observation device according to the present disclosure are useful for measuring elastic properties properly by combining the strain method with the shear wave method.

The present disclosure affords the advantageous effect of combining the strain method with the shear wave method to enable elastic properties to be measured properly.

Additional advantages and modifications will readily occur to those skilled in the art. Therefore, the disclosure in its broader aspects is not limited to the specific details and representative embodiment shown and described herein. Accordingly, various modifications may be made without departing from the spirit or scope of the general concept as defined by the appended claims and their equivalents.

What is claimed is:

1. An ultrasound observation device configured to acquire an echo signal which is obtained by converting, to an electrical signal, ultrasound received by an ultrasound probe by using a first measurement method that utilizes displacement of an observation target to measure elasticity information of the observation target, and a second measurement method that utilizes a shear wave generated by the observation target to measure the elasticity information of the observation target, the ultrasound observation device comprising
a processor configured to control the ultrasound observation device and the ultrasound probe, wherein the processor is configured to
control the ultrasound probe to execute the second measurement method based on the echo signal obtained using the first measurement method.

2. The ultrasound observation device according to claim 1, wherein the processor is configured to:
determine whether or not a reference condition of the first measurement method is fulfilled by using a plurality of temporally preceding or subsequent images based on the echo signal, which are two images based on the echo signal obtained using the first measurement method;
determine, in a case where it is determined that the reference condition is fulfilled, whether or not an execution condition of the second measurement method is fulfilled by using a plurality of temporally preceding or subsequent images based on the echo signal, which are a plurality of images based on the echo signal obtained using the first measurement method; and
control the ultrasound probe to execute the second measurement method in a case where it is determined that the execution condition is fulfilled.

3. The ultrasound observation device according to claim 2, wherein the processor is configured to:
determine whether or not the observation target has been substantially fixed; and
determine whether or not the reference condition is fulfilled when it is determined that the observation target has been substantially fixed.

4. The ultrasound observation device according to claim 3, wherein the processor is configured to:
determine whether or not the reference condition is fulfilled based on at least any one of the colored amount of the elasticity image corresponding to the elasticity information obtained using the first measurement method, the periodicity of the displacement of the observation target, and the displacement direction;
set a reference image for use in the determination of whether the execution condition is fulfilled; and
determine whether or not the execution condition is fulfilled based on the matching degree between the reference image and the determination-object image, and the displacement amount or the movement amount of the observation target.

5. The ultrasound observation device according to claim 4, wherein the processor is configured to determine that the reference condition is fulfilled when at least one of the following conditions is fulfilled: the surface area of a colored region of the elasticity image corresponding to the elasticity information obtained using the first measurement method is greater than or equal to a reference colored surface area; the volatility of the recurring duration of the period of the displacement of the observation target is less than or equal to a reference volatility; and the displacement direction is perpendicular to the scanning direction of the ultrasound.

6. The ultrasound observation device according to claim 5, wherein the processor is configured to determine that the execution condition is fulfilled when the matching degree between the reference image and the determination-target image is greater than or equal to a reference matching degree, and the displacement amount is less than or equal to a reference displacement amount or the movement amount is less than or equal to a reference movement amount.

7. The ultrasound observation device according to claim 4, wherein the processor is configured to set, as the reference image, an image which is used upon determining that the reference condition is fulfilled.

8. The ultrasound observation device according to claim 4, wherein the processor is configured to use the displacement amount to predict the timing at which the displacement amount becomes zero.

9. The ultrasound observation device according to claim 4, wherein the processor is configured to use the period of displacement of the observation target to predict the timing at which the displacement amount peaks.

10. An operating method of an ultrasound observation device configured to acquire an echo signal which is obtained by converting, to an electrical signal, ultrasound received by an ultrasound probe by using a first measurement method that utilizes displacement of an observation target to measure elasticity information of the observation target, and a second measurement method that utilizes a shear wave generated by the observation target to measure the elasticity information of the observation target, the operating method comprising:
determining whether or not a reference condition of the first measurement method is fulfilled by using a plurality of temporally preceding or subsequent images based on the echo signal, which are a plurality of images based on the echo signal obtained using the first
determining, in a case where it is determined in the second determination step that the reference condition is fulfilled, whether or not an execution condition of the second measurement method is fulfilled by using a plurality of temporally preceding or subsequent images based on the echo signal, which are a plurality of images based on the echo signal obtained using the first measurement method; and
controlling the ultrasound probe to execute the second measurement method in a case where it is determined in the third determination step that the execution condition is fulfilled.

11. A non-transitory computer-readable recording medium on which an executable program is recorded, the program causing a processor of an ultrasound observation device to execute, the ultrasound observation device being configured to acquire an echo signal which is obtained by converting, to an electrical signal, ultrasound received by an ultrasound probe by using a first measurement method that utilizes displacement of an observation target to measure elasticity information of the observation target, and a second measurement method that utilizes a shear wave generated by the observation target to measure the elasticity information of the observation target:
determining whether or not a reference condition of the first measurement method is fulfilled by using a plurality of temporally preceding or subsequent images based on the echo signal, which are a plurality of images based on the echo signal obtained using the first
determining, in a case where it is determined in the second determination step that the reference condition is fulfilled, whether or not an execution condition of the second measurement method is fulfilled by using a plurality of temporally preceding or subsequent images based on the echo signal, which are a plurality of images based on the echo signal obtained using the first measurement method; and
controlling the ultrasound probe to execute the second measurement method in a case where it is determined in the third determination step that the execution condition is fulfilled.
